(19) United States
(12) Patent Application Publication
(10) Pub. No.: US 2006/0016698 A1
(43) Pub. Date: Jan. 26, 2006

(54) Method and apparatus for electrochemical detection

(76) Inventors: Chih-Kung Lee, Potomac, MD (US); Wen-Jong Wu, Taipei County (TW); Wen-Hsin Hsiao, Taoyuan County (TW)

Correspondence Address: AKIN GUMP STRAUSS HAUER & FELD L.L.P., ONE COMMERCE SQUARE, 2005 MARKET STREET, SUITE 2200, PHILADELPHIA, PA, 19103, US

(21) Appl. No.: 11/038,121

(22) Filed: Jan. 21, 2005

(30) Foreign Application Priority Data
Jul. 22, 2004 (TW) 093121861

(51) Int. Cl. G01N 27/26; G01N 33/487

(52) U.S. Cl. 205/777.5; 205/792; 204/403.01

(57) ABSTRACT

An apparatus for quantitatively determining an analyte in a sample fluid includes a holder for holding an electrochemical cell that includes a catalyst, a waveform generator for generating a potential profile having a voltage bias and an alternating part, a detector for detecting a current signal for a period of measuring time through the electrochemical cell, a memory for storing the current signal, and a processor for correlating the current signals with the concentration of the analyte.

This application claims the benefit of Taiwan Application No. 093121861, filed Jul. 22, 2004, which is herein incorporated by reference in its entirety.

### BACKGROUND

I. Field of the Invention

The present invention relates generally to electrochemical detection, and, more particularly, to a method and apparatus for quantitatively determining the concentration of an analyte in a fluid sample.

II. Background of the Invention

In the field of biomedical techniques, biosensors have been developed to analyze human body fluids in order to diagnose potential diseases or monitor health condition. A biosensor is an analytical device that comprises at least a biological component for selective recognition of an analyte in a sample fluid and a transducer device for relaying biological signals for further analysis. For example, biosensors are typically used to monitor lactate, cholesterol, bilirubin and glucose in certain individuals. In particular, determination of the concentration of glucose in body fluids such as blood is of great importance to diabetic individuals, who must frequently check the level of glucose in their blood as a means of regulating the glucose intake in their diets and monitoring the effects of therapeutics. With proper maintenance of blood glucose through daily injections of insulin and strict control of dietary intake, the prognosis for diabetics is excellent for type-I patients. Since blood glucose levels must be closely followed in diabetic individuals, an ideal biosensor for the detection of glucose must be simple and easy to operate without compromising accuracy.

In electrochemistry, an interplay between electricity and chemistry concerns current, potential, and charge from an electrochemical reaction. There are generally two types of electrochemical measurements, potentiometric and amperometric. The potentiometric technique is a static technique with no current flow, which has been widely used for monitoring ionic species such as calcium, potassium, and fluoride ions. The amperometric technique is used to drive an electron-transfer reaction by applying a potential. A responsive current measured is related to the presence and/or concentration of a target analyte. Amperometric biosensors make possible a practical, fast, and routine measurement of test analyte.

The success in the development of the amperometric devices has led to amperometric assays for several biomolecules including glucose, cholesterol, and various drugs. In general, an amperometric biosensor includes an insulating base plate, two or three electrodes, a dielectric layer, and a region containing an enzyme as a catalyst and at least one redox mediator for introduction of electron-transfer during the enzymatic oxidation of the analyte. The reaction progresses when a sample liquid containing an analyte is added onto the reaction region. Two physical effects, mesh spread and capillary action, are commonly used to guide a uniform distribution of the applied sample on the reaction region. A controlled potential is then applied between the electrodes to trigger oxidoreduction. The test analyte is therefore oxidized and electrons are generated from the accompanying chain reaction of the enzyme and mediator. The applied electrical potential must be sufficient enough to drive a diffusion-limited electrooxidation, yet insufficient to activate irrelevant chemical reactions. After a short time of delay, the current generated by the electrochemical oxidoreduction is observed and measured and the current is correlated to the presence and/or amount of the analyte in the sample.

Examples of conventional techniques for amperometric detection can be found in U.S. Pat. No. 5,620,579 to Genshaw et al., entitled “Apparatus for Reduction of Bias in Amperometric Sensors” (hereinafter “the '579 patent”), and U.S. Pat. No. RE. 36,268 to Szuminsky et al., entitled “Method and Apparatus for Amperometric Diagnostic Analysis” (hereinafter “the '268 patent.) Each of these references proposes a different way to supply the potential to trigger the electrochemistry reaction. The '579 patent discloses a method for determining the concentration of an analyte by applying a first potential, which is a burn-off voltage potential, to an amperometric sensor and then applying a second potential, which is a read voltage potential, to the amperometric sensor. A first current in response to the burn-off voltage potential and a second current in response to the read voltage potential are measured for calculating a bias correction value in order to enhance the accuracy of the analyte determination.

The '268 patent discloses a method for quantitatively determining biologically important compounds in body fluids. The '268 patent does not provide any voltage at an early stage of electrochemical reaction, avoiding unwanted power consumption at the early stage. After a span of time, a constant voltage is applied to a sample and a corresponding Cottrell current is measured.

The trend of new generations of biosensors focuses on the methodology of quick response time and higher resolution. It is desirable to have an apparatus or method for electrochemical detection that can achieve improved signal resolution and efficient power consumption for detection. It is also desirable to achieve detection by modifying the profile of the potential supplied to trigger the electrochemistry reaction.

### BRIEF SUMMARY OF THE INVENTION

The present invention is directed to an apparatus and method that may enhance electrochemical reaction and achieve improved signal resolution. The present invention proposes a potential profile that comprises a voltage bias and an alternating part such as a sinusoidal wave to trigger the electrochemistry reaction. By supplying the potential profile, the electrochemical reaction is enhanced and results in improved signal resolution. In accordance with an embodiment of the present invention, there is provided a method for quantitatively determining an analyte that comprises adding a sample fluid containing an analyte to an electrochemical cell that includes an enzyme, applying a potential profile to the electrochemical cell, measuring a current signal for a period of measuring time through the electrochemical cell, and correlating the current signals with the concentration of the analyte.

Further in accordance with the present invention, there is provided an apparatus for measuring the amount of an analyte in a sample fluid that comprises a holder for holding an electrochemical cell that includes a catalyst, a waveform generator for generating a potential profile, wherein the potential profile comprises a voltage bias and an alternating part, a detector for detecting a current signal for a period of measuring time through the electrochemical cell, a memory for storing the current signal detected in the period of measuring time, and a processor for correlating the current signal with a concentration of the analyte.

Additional features and advantages of the present invention will be set forth in part in the description which follows, and in part will be obvious from the description, or may be learned by practice of the invention. The features and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

The accompanying drawings, which are incorporated in and constitute a part of this specification, illustrate one embodiment of the present invention and together with the description, serves to explain the principles of the invention.

### BRIEF DESCRIPTION OF THE DRAWINGS

Reference will now be made in detail to the present embodiment of the invention, an example of which is illustrated in the accompanying drawings.

Wherever possible, the same reference numbers are used throughout the drawings to refer to the same or like parts.

FIG. 1 is a block diagram of a system for determining the concentration of an analyte contained in a sample fluid in accordance with one embodiment of the present invention;

FIG. 2 is a schematic diagram of an apparatus for measuring the concentration of an analyte in accordance with one embodiment of the present invention;

FIG. 3A is a plot showing an experimental result of applying a constant voltage to a sample fluid containing an analyte at various concentration levels;

FIG. 3B is a plot showing an experimental result of applying a potential profile to a sample fluid containing an analyte at various concentration levels in accordance with one embodiment of the present invention;

FIG. 3C is a plot showing a comparison between experimental results of applying to a sample fluid a constant voltage and a potential profile;

FIG. 4 is a plot illustrating methods for processing a current signal in accordance with one embodiment of the present invention; and

FIG. 5 is a flow diagram showing a method for correlating a current signal with a concentration of an analyte in accordance with one embodiment of the present invention.

### DETAILED DESCRIPTION OF THE INVENTION

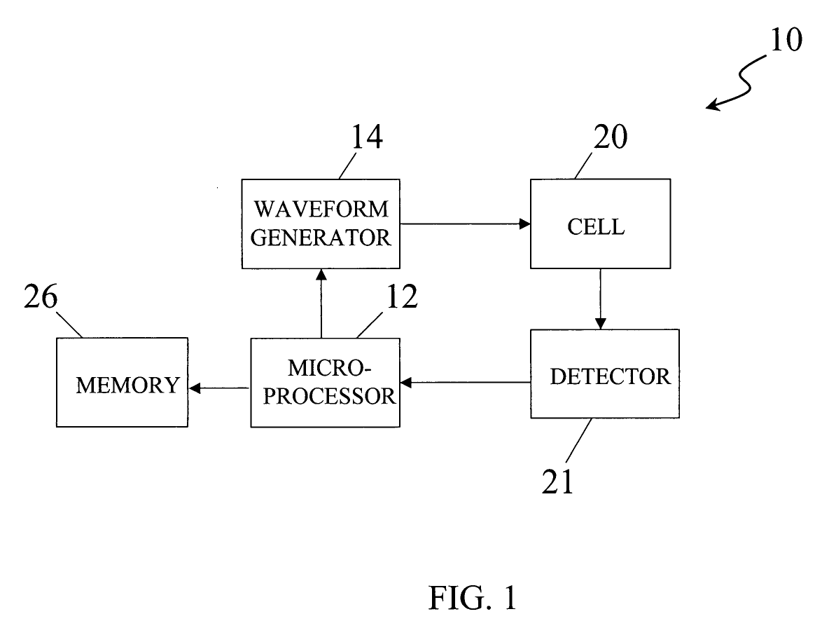

FIG. 1 is a block diagram of a system 10 for determining the concentration of an analyte in a sample fluid in accordance with one embodiment of the present invention. The sample fluid includes, but not limited to, blood, lymph, saliva, vaginal and anal secretions, urine, feces, perspiration, tears, and other bodily fluids. Referring to FIG. 1, system 10 includes a microprocessor 12, a waveform generator 14, a cell 20, a detector 21, and a memory 26.

A potential profile is set to trigger an electrochemical reaction in cell 20. The potential profile comprises a voltage bias and an alternating part. The alternating part, having an amplitude and transmitting at a frequency, includes one of a sinusoidal wave, a triangular wave, a square wave, or a combination thereof. A volume of a test sample containing an analyte of a concentration is added to cell 20. Microprocessor 12, in response to the application of the test sample, enables waveform generator 14 to generate a potential in accordance with the designed profile. Various commercially available data acquisition apparatuses, such as a DAQ card manufactured by National Instruments (Austin, Tex.), can be used as waveform generator 14. In one embodiment according to the present invention, a potential profile comprises a voltage bias of 0.4V (volts) and an alternating part, which is a sinusoidal wave having an amplitude of 0.1V and a frequency of 1 Hz (Hertz), in the case where glucose is selected as the analyte. In one aspect, the voltage bias includes a direct-current (dc) component having a constant value over a measuring period. In another aspect, the voltage bias includes a dc component which is time-varying over a measuring period. Moreover, in other embodiments according to the present invention where glucose is selected as the analyte, the voltage bias may have a value, either constant or time-varying, ranging from approximately 0.1V to 1.0V, and the sinusoidal wave may have an amplitude ranging from approximately 0.0V to 0.5V at a frequency ranging from 0.5 Hz to 100 Hz. The voltage bias, amplitude and frequency may change as cell 20 changes.

Although the embodiment directed towards the determination of glucose is discussed, skilled persons in the art will understand that the method and apparatus of the present invention can be used for the determination of other analytes upon selection of an appropriate catalyst such as an enzyme. Examples of the analytes include a substance metabolite such as glucose, cholesterol, triglyceride or latic acid, a hormone such as T4 or TSH, a physiological constituent such as albumin or hemoglobin, a biomarker including protein, lipid, carbohydrate, deoxyribonucleic acid or ribonucleic acid, a drug such as an antiepileptic or an antibiotic, or a non-therapeutic compound such as a heavy metal or toxin.

The potential profile generated by waveform generator 14 is applied to cell 20. Cell 20, an electrochemical cell where the electrochemical reaction takes place, contains an enzyme, which has been previously applied thereto. The electrochemical reaction occurs via at least one electron transfer agent. Given a biomolecule A, the oxidoreductive process is described by the following reaction equation:

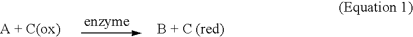

The biomolecule A is oxidized to B by an electron transfer agent C, in the presence of an appropriate enzyme. Then the electron transfer agent C is oxidized at an electrode of cell 20
C (red)→C(ox)+n e⁻ (Equation 2)
where n is an integer. Electrons are collected by the electrode and a resulting current is measured.

Those skilled in the art will recognize there are many different reaction mechanisms that will achieve the same result. Equations 1 and 2 are non-limiting examples of such a reaction mechanism.

As an example, a glucose molecule and two ferricyanide anions in the presence of glucose oxidase produce gluconolacton, two ferrocyanide anions, and two protons by the following equation:

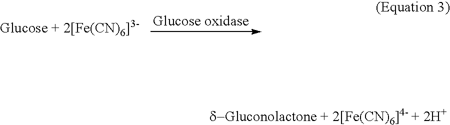

The amount of glucose present is assayed by electrooxidizing the ferrocyanide anions to ferricyanide anions and measuring the charge passed. The process mentioned above is described by the following equation:
[Fe(CN)₆]⁴⁻→[Fe(CN)₆]³⁻+e⁻ (Equation 4)

In a preferred embodiment of the invention, an appropriate enzyme for glucose is glucose oxidase, and the reagent in electrochemical cell 20 contains the following formulations: 600 u/ml of glucose oxidase, 0.4M of potassium ferricyanide, 0.1M of phosphate buffer, 0.5M of potassium chloride, and 2.0 g/dl of gelatin.

In another example, the amount of total cholesterol contained in a sample fluid, which may include cholesterol and cholesterol esters, is to be measured. Appropriate enzymes provided in cell 20 include cholesterol esterase and cholesterol oxidase. The cholesterol esters are hydrolyzed to cholesterol in the presence of cholesterol esterase, as given in an equation below.

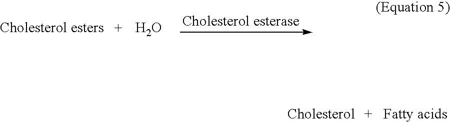

The cholesterol is then oxidized to cholestenone, as given in an equation below.

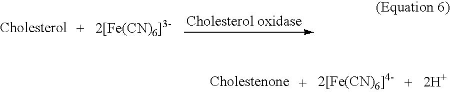

The amount of total cholesterol is assayed by electrooxidizing the ferrocyanide anions to ferricyanide anions and measuring the charge passed.
[Fe(CN)₆]³¹ →[Fe(CN)₆]³⁻+e⁻ (Equation 7)

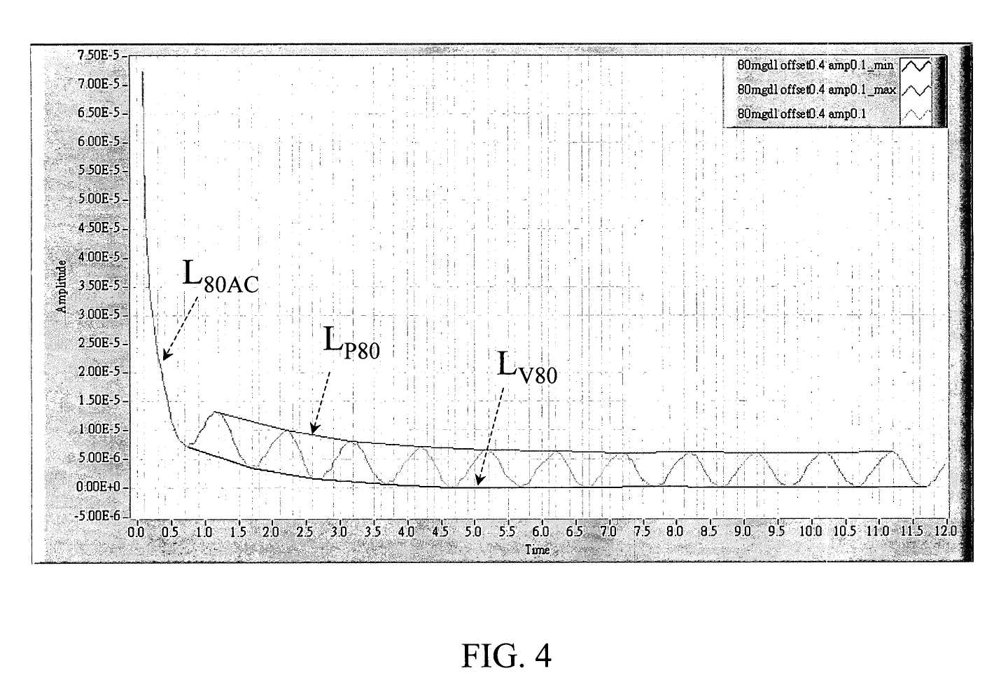

Detector 21 detects an output current signal from cell 20. Microprocessor 12 processes and analyzes the current signal, and correlates the processed current signal with the concentration of glucose. Methods for processing the current signal will be discussed in detail with reference to FIG. 4. Memory 26 stores the processed data and a current-concentration relationship under the same potential profile. System 10 may further include a display device (not shown) for display of the detection result.

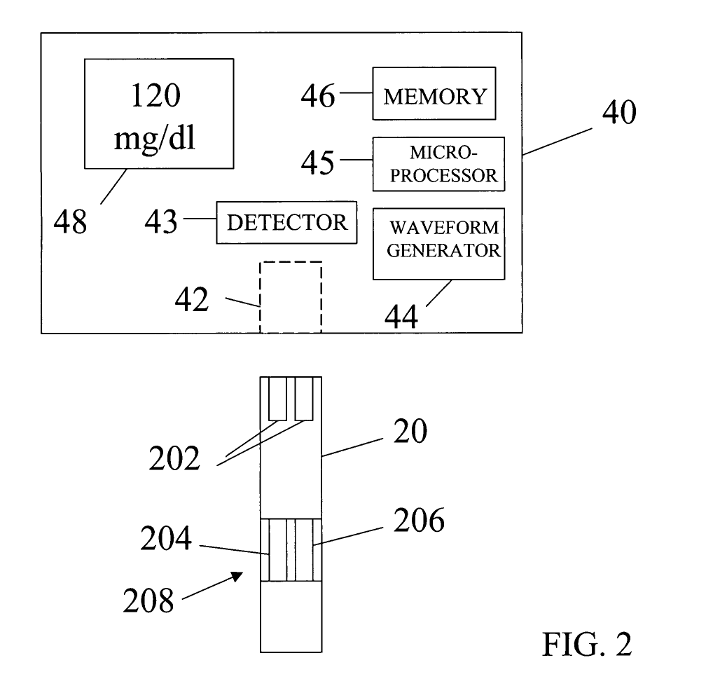

FIG. 2 is a schematic diagram of an apparatus 40 for measuring the concentration of an analyte in accordance with one embodiment of the present invention. Referring to FIG. 2, apparatus 40 includes a holder 42, a detector 43, a waveform generator 44, a microprocessor 45 and a memory 46. Holder 42 receives and holds cell 20. Memory 46 has been stored with, for example, a lookup table that specifies the concentration-current relationship between various concentrations of an analyte and corresponding current levels. Waveform generator 44 generates a potential profile having substantially the same profile as those used for establishing the concentration-current relationship. The potential profile is applied to cell 20. Detector 43 detects a current signal provided from cell 20. Microprocessor 45 processes the current signal and correlating the processed result with the concentration.

Cell 20 to be inserted to apparatus 40 includes conductive contacts 202, and electrodes 204 and 206 electrically connected (not shown) to conductive contacts 202. Electrodes 204 and 206 are disposed at a reaction region 208, where an appropriate catalyst such as an enzyme for an analyte has been provided. When a sample liquid containing an analyte is added to cell 20 at reaction region 208, the reaction involving the analyte and an electron transfer agent proceeds as previously described with respect to Equations 1 and 2. Later, when the potential profile from waveform generator 44 is applied to cell 20, a current flow, generated as previously described with respect to Equations 2 and 4, is detected by apparatus 40. The detected current level is compared with the lookup table stored in memory 46 by mapping, linear interpolation or other methods. An indicator 48 of apparatus 40 displays the glucose level for the sample liquid.

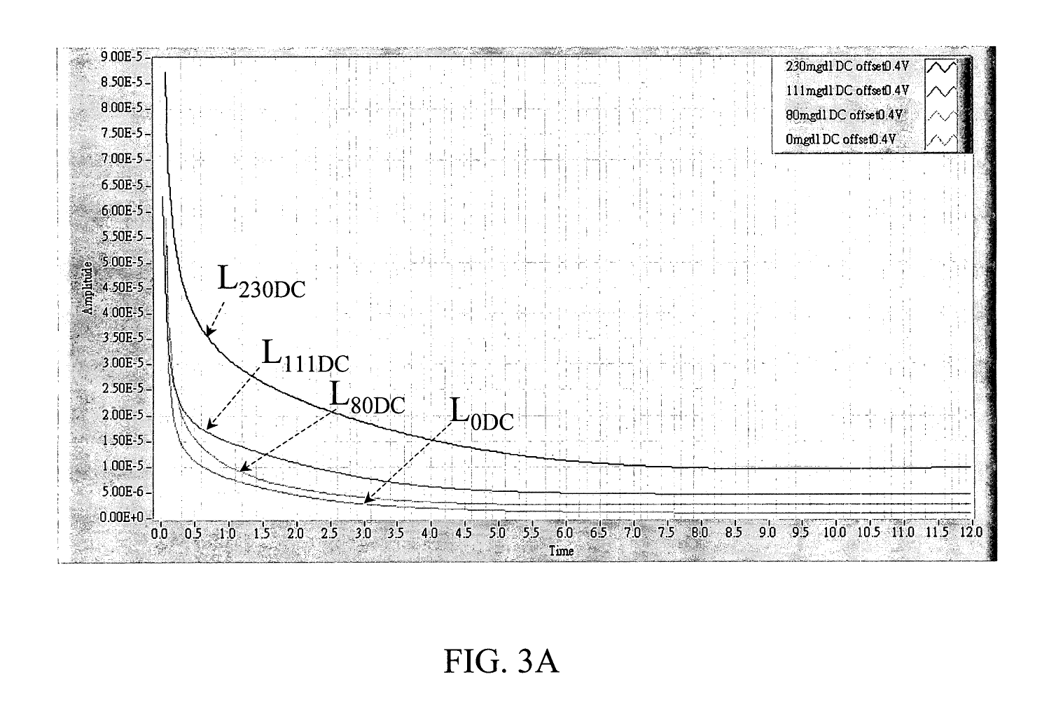

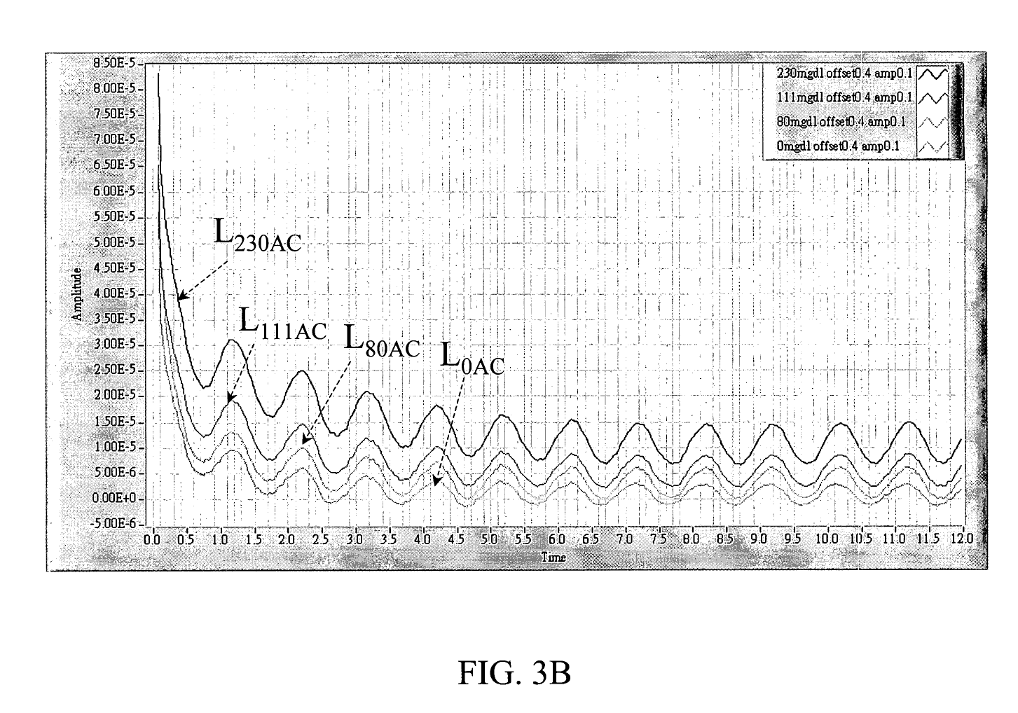

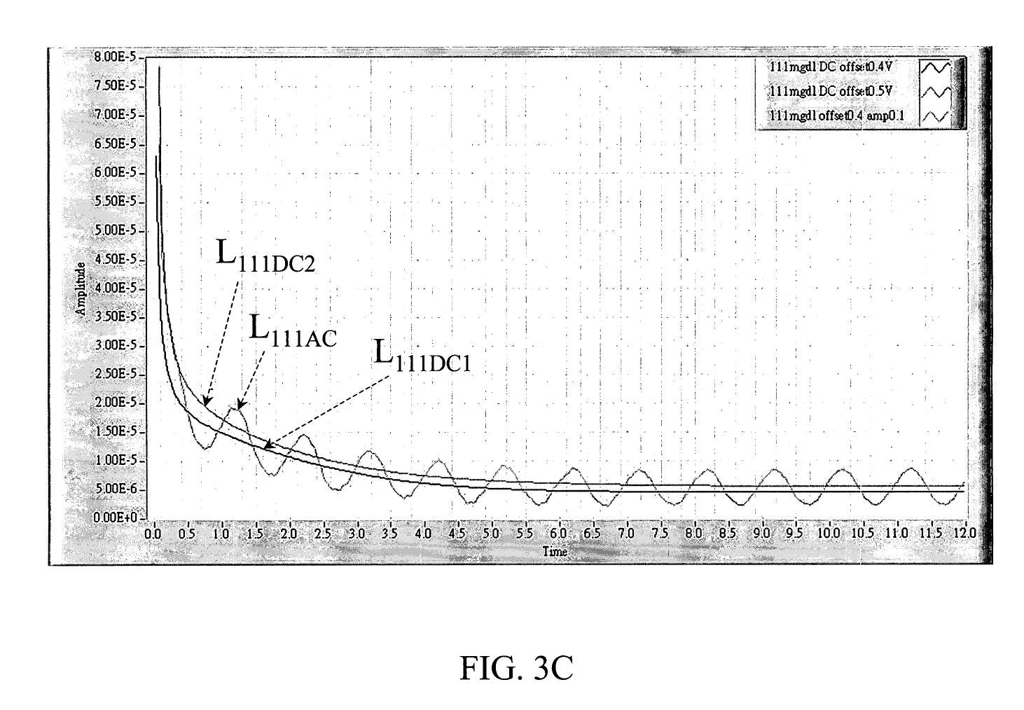

FIG. 3A is a plot showing an experimental result of applying a constant voltage to a sample fluid containing an analyte at various concentrations. Referring to FIG. 3A, a constant voltage of 0.4V is applied to sample fluids containing glucose at the concentrations of 230 mg/dl, 111 mg/dl, 80 mg/dl and 0 mg/dl, respectively. The glucose concentration of these sample fluids are determined by a colometric method based upon the reactions:
Glucose+O₂+H₂O→Gluconic acid+H₂O₂
H₂O₂+Reagent H₂O+Red dye

Response currents are represented by curves L_{230DC}, L_{111DC}, L_{80DC} and L_{0DC}. At an early stage, for example, from 0 to 0.5 second, an unstable current may occur due to an unstable electrochemical reaction. Moreover, the magnitude of a response current decreases over time as the electrochemical reaction proceeds.

FIG. 3B is a plot showing an experimental result of applying a potential profile to a sample fluid containing an analyte at various concentrations in accordance with one embodiment of the present invention. Referring to FIG. 3B, a potential profile that comprises a voltage bias of 0.4V and a sinusoidal wave having an amplitude of 0.1V and a frequency of 1 Hz is applied to electrochemical cells that include glucose at the concentrations of 230 mg/dl, 111 mg/dl, 80 mg/dl and 0 mg/dl, respectively.

Response currents are represented by curves L_{230AC}, L_{111AC}, L_{80AC} and L_{0AC}. According to American Diabetics Association (“ADA”), blood glucose normally falls between 50 to 100 mg/dl before meal, and rises up to a level generally less than 170 mg/dl after meal. The selected range, 0 to 230 mg/dl, which may be directed to diabetic individuals, is wider than the normal range suggested by ADA.

FIG. 3C is a plot showing a comparison between experimental results of applying to a sample fluid a constant voltage and a potential profile. Referring to FIG. 3C, curves L_{111DC1} and L_{111DC2} represent response current signals measured by applying constant voltages of 0.4V and 0.5V, respectively, to a sample fluid containing glucose of 111 mg/dl, and a curve L_{111AC} represents a response current signal measured by applying a potential profile that comprises a voltage bias of 0.4V and a sinusoidal wave having an amplitude of 0.1V and a frequency of 1 Hz to an electrochemical cell that includes glucose of 111 mg/dl. It can be seen that the curve L_{111AC} has a higher current response, and in turn a higher resolution, than the curves L_{111DC1} and L_{111DC2}. In particular, when the curves L_{111AC} and L_{111DC2} are compared to one another, the curve L_{111AC} has a higher resolution than the curve L_{111DC2}, which means that the method using the potential profile is advantageous.

FIG. 4 is a plot illustrating methods for processing a current signal in accordance with one embodiment of the present invention. Referring to FIG. 4, as an example of the curve L_{80AC} shown in FIG. 3B, the peaks of the curve L_{80AC} are connected to form a peak curve L_{P80} by, for example, curve fitting. In another aspect, the valleys of the curve L_{80AC} are connected to form a valley curve L_{V80}. To correlate the current signal with a concentration of the analyte, i.e., glucose, in a first example, the current magnitude of a peak curve of a response curve is measured at a time point during a measuring period of approximately 60 seconds. The time point should be selected from a stable current region of the response curve without the concern of any unstable reaction. In a second example, the current magnitude of a valley curve of a response curve is measured at a time point. The first and second examples as an example of response curves L_{0AC}, L_{80AC}, L_{111AC} and L_{230AC} are summarized in Table 1.

Table 1 shows experimental results of methods for correlating current signals with the amount of the analyte in the sample fluid. Specifically, the second and third columns of Table 1 refer to methods in accordance with the above-mentioned first and second examples of the present invention, respectively, where the current magnitudes are taken at the fourth second once the potential profile (the same as that shown in FIG. 3B) is applied. By comparison, the last column of Table 1 refers to a method for measuring the current magnitude at the fourth second once a constant voltage is applied.

TABLE 1

| | | | Current magnitude of |
|---|---|---|---|
| | Current magnitude of | Current magnitude of | a response curve at |
| | a peak curve of a | a valley curve of a | the fourth second |
| Concentration of | response curve at the | response curve at the | under a constant |
| glucose (mg/dl) | fourth second (μA) | fourth second (μA) | voltage of 0.4 V (μA) |
| 0 | 3.89 | −1.19 | 1.60 |
| 80 | 6.88 | 0.46 | 3.72 |
| 111 | 9.75 | 2.87 | 7.38 |
| 230 | 17.62 | 9.24 | 14.91 |

Moreover, in a third example, a response curve is integrated over a time period to calculate the amount of charges. In a fourth example, a peak curve of a response curve is integrated over a time period to calculate the amount of charges. In a fifth example, a valley curve of a response curve is integrated over a time period to calculate the amount of charges. The operations such as curve fitting and integration may be performed in microprocessor 12. The third, fourth and fifth examples as an example of response curves L_{0AC}, L_{80AC}, L_{111AC} and L_{230AC} are summarized in Table 2.

Table 2 shows experimental results of other methods for correlating current signals with the amount of the analyte. Specifically, the second, third and fourth columns of Table 2 refer to methods in accordance with the above-mentioned third, fourth and fifth embodiments of the present invention, respectively, where the curves are integrated over a time period from the first to the sixth second once the potential profile is applied. By comparison, the last column of Table 2 refers to a method for integrating response curves over the same period once a constant voltage is applied.

TABLE 2

| | | | | Amount of |
|---|---|---|---|---|
| | | | | charges |
| | | Amount of | Amount of | calculated by |
| | Amount of | charges | charges | integrating a |
| | charges | calculated by | calculated by | response curve |
| | calculated by | integrating a peak | integrating a | from the first to |
| | integrating a | curve of a | valley curves of a | sixth second |
| Concentration | response curve | response curve | response curve | under a constant |
| of glucose | from the first to | from the first to | from the first to | voltage of 0.4 V |
| (mg/dl) | sixth second (Q) | sixth second (Q) | sixth second (Q) | (Q) |
| 0 | 10.79 | 22.93 | −1.10 | 14.57 |
| 80 | 24.23 | 40.24 | 8.60 | 28.16 |
| 111 | 41.41 | 58.89 | 25.98 | 44.07 |
| 230 | 81.13 | 103.34 | 60.96 | 88.79 |

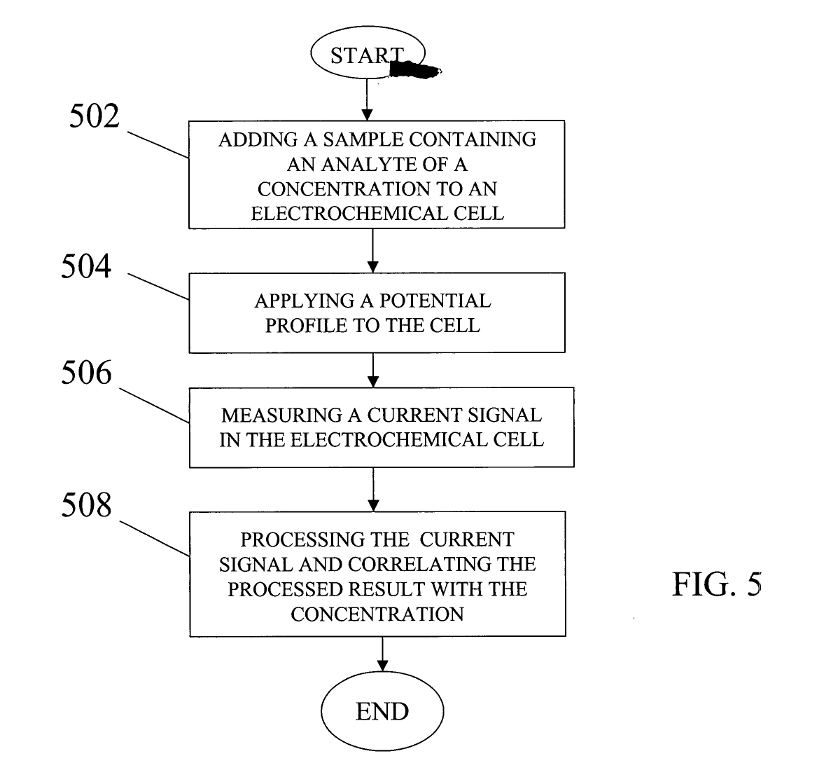

FIG. 5 is a flow diagram showing a method for correlating a current signal with a concentration of an analyte in accordance with one embodiment of the present invention. Referring to FIG. 5, a sample containing an analyte of a concentration is applied to a cell 20 at step 502. Next, a potential profile including a voltage bias and an alternating part is applied to the sample at step 504. A response current signal is then measured at step 506. Microprocessor 12 processes the response current to derive a concentration-current relationship for the analyte at step 508. In processing the response current, the methods in accordance with the present invention as previously described with respect to Table 1 and Table 2 may be used. The concentration-current relationship may be stored in memory 46 in the form of a lookup table.

The foregoing disclosure of the preferred embodiments of the present invention has been presented for purposes of illustration and description. It is not intended to be exhaustive or to limit the invention to the precise forms disclosed. Many variations and modifications of the embodiments described herein will be apparent to one of ordinary skill in the art in light of the above disclosure. The scope of the invention is to be defined only by the claims appended hereto, and by their equivalents.

Further, in describing representative embodiments of the present invention, the specification may have presented the method and/or process of the present invention as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process of the present invention should not be limited to the performance of their steps in the order written, and one skilled in the art can readily appreciate that the sequences may be varied and still remain within the spirit and scope of the present invention.

What is claimed is:

1. A method for quantitatively determining an analyte in a sample fluid, comprising:
adding a sample fluid containing an analyte to an electrochemical cell that includes at least one catalyst;
applying a potential profile to the electrochemical cell, wherein the potential profile comprises a voltage bias and an alternating part;
measuring a current signal for a period of measuring time through the electrochemical cell; and
correlating the current signal with an amount of the analyte in the sample fluid.

2. The method of claim 1, wherein the alternating part includes one of a sinusoidal, triangular or square wave.

3. The method of claim 1, wherein the alternating part includes a combination of a sinusoidal, triangular or square wave.

4. The method of claim 1, wherein the voltage bias includes a direct-current (dc) component having a constant value.

5. The method of claim 1, wherein the voltage bias includes a dc component having a time-varying value.

6. The method of claim 1, further comprising:
integrating the current signal over a period of time to calculate an amount of charges; and
correlating the amount of charges with the concentration of the analyte in the sample fluid.

7. The method of claim 1, further comprising:
connecting the peaks of the current signal in the period of measuring time to generate a curve;
determining a magnitude of the curve at a time point in the period of measuring time; and
correlating the magnitude with the concentration of the analyte in the sample fluid.

8. The method of claim 1, further comprising:
connecting the valleys of the current signal in the period of measuring time to generate a curve;
determining a magnitude of the curve at a time point in the period of measuring time; and
correlating the magnitude with the concentration of the analyte in the sample fluid.

9. The method of claim 1, further comprising:
connecting the peaks of the current signal in the period of measuring time to generate a curve;
integrating the curve over a period of time to calculate an amount of charges; and
correlating the amount of charges with the concentration of the analyte in the sample fluid.

10. The method of claim 1, further comprising:
connecting the valleys of the current signal in the period of measuring time to generate a curve;
integrating the curve over a period of time to calculate an amount of charges; and
correlating the amount of charges with the concentration of the analyte in the sample fluid.

11. The method of claim 1, wherein the period of measuring time is in the range of approximately 0.5 to 60 seconds.

12. The method of claim 1, wherein the analyte is glucose, and the at least one catalyst includes glucose oxidase.

13. The method of claim 1, wherein the analyte includes at least one of cholesterol or cholesterol esters, and the at least one catalyst includes cholesterol oxidase.

14. The method of claim 1, wherein the analyte includes one of a substance metabolite, hormone, physiological consitituent, biomarker, drug or non-therapeutic compound.

15. The method of claim 14, wherein the analyte includes one of triglyceride, latic acid, T4, TSH, albumin, hemoglobin, protein, carbohydrate, lipid, deoxyribonucleic acid, ribonucleic acid, antiepileptic, antibiotic, heavy metal or toxin.

16. An apparatus for quantitatively determining an analyte in a sample fluid, comprising:
a holder for holding an electrochemical cell that includes at least one catalyst;
a voltage generator for generating a potential profile, wherein the potential profile comprises a voltage bias and an alternating part;
a detector for detecting a current signal for a period of measuring time through the electrochemical cell;
a memory for storing the current signal; and
a processor for correlating the current signal with a concentration of the analyte.

17. The apparatus of claim 16, wherein the alternating part includes one of a sinusoidal, triangular or square wave.

18. The apparatus of claim 16, wherein the voltage bias includes a direct-current (dc) component having a constant value over the period of measuring time.

19. The apparatus of claim 16, wherein the voltage bias includes a dc component having a time-varying value over the period of measuring time.

20. The apparatus of claim 16, wherein the alternating part includes a combination of a sinusoidal, triangular or square wave.

21. The apparatus of claim 16, wherein the period of measuring time is in the range of approximately 0.5 to 60 seconds.

22. The apparatus of claim 16, wherein the analyte is glucose, and the at least one catalyst includes glucose oxidase.

23. The apparatus of claim 16, wherein the analyte includes one of cholesterol or cholesterol esters, and the at least one catalyst includes cholesterol oxidase.

24. The apparatus of claim 16, wherein the analyte includes one of a substance metabolite, hormone, physiological consitituent, biomarker, drug or non-therapeutic compound.

25. An apparatus for quantitatively determining glucose in a sample fluid, comprising:
a holder for holding an electrochemical cell that includes glucose oxidase;
a voltage generator for generating a potential profile; wherein the potential profile comprises a voltage bias and an alternating part;
a detector for detecting a current signal generated in response to the potential profile for a period of measuring time through the electrochemical cell;
a memory for storing the current signal; and
a processor for correlating the current signal with the concentration of the analyte.

26. The apparatus of claim 25, wherein the potential profile comprises a voltage bias ranging from approximately 0.1V to 1.0V.

27. The apparatus of claim 25, wherein the potential profile comprises a sinusoidal wave having an amplitude ranging from approximately 0.01V to 0.5V.

28. The apparatus of claim 25, wherein the potential profile comprises a sinusoidal wave having a frequency ranging from approximately 0.5 Hz to 100 Hz.
